(19) United States
(12) Patent Application Publication
(10) Pub. No.: US 2008/0203185 A1
(43) Pub. Date: Aug. 28, 2008

(54) Volatile Liquid Disseminating Device

(76) Inventors: Colin Brown, Bracknell Berkshire (GB); Guy Edward Naish, Bicester Oxfordshire (GB); Kishen Gohil, New Malden Surrey (GB)

Correspondence Address: CURATOLO SIDOTI CO., LPA, 24500 CENTER RIDGE ROAD, SUITE 280, CLEVELAND, OH, 44145, US

(21) Appl. No.: 10/570,039

(22) PCT Filed: Aug. 20, 2004
(86) PCT No.: PCT/CH2004/000526

§ 371 Date: Apr. 16, 2008

(30) Foreign Application Priority Data
Sep. 2, 2003 (GB) 0320461.7

(51) Int. Cl. A61L 9/12

(52) U.S. Cl. 239/44

(57) ABSTRACT

An apparatus for disseminating a volatile liquid, such as a fragrance, into an atmosphere from a reservoir, dissemination being achieved by means of a transfer member in contact with the liquid and a capillary member in liquid transfer contact with the transfer member. The capillary member comprises an evaporating surface bearing primary capillary channels, at least some of these primary capillary channels being intersected by at least one secondary capillary channel, the secondary capillary channel being substantially smaller in cross-sectional area than that of the primary channels, such that liquid will flow in both primary and secondary channels.

This invention relates to capillary members for use in apparatus adapted to disseminate volatile liquids into an atmosphere.

A common method of disseminating volatile liquids, such as fragrances and insecticides, into an atmosphere is by evaporation from a porous transfer member, such as a fibrous wick, which is in contact with a reservoir of volatile liquid. This system has drawbacks, such as the low surface area for evaporation and the tendency for fractionation of liquids that are mixtures. It has been proposed to fit to such a transfer member a capillary member, in the form of a sheet that extends essentially perpendicularly from the transfer member and that comprises channels of capillary dimensions, to which volatile liquid can pass and travel along for evaporation. This sheet generally contacts the transfer member by means of a hole in the sheet through which the transfer member protrudes and within which it fits snugly, at least some of the capillary channels being in contact with the transfer member, such that liquid can transfer from the member to the sheet (“liquid transfer contact”).

A further type of capillary member is described in U.S. Pat. No. 4,913,350. In this case, a capillary member is inserted directly into the liquid without the need for a transfer member. Such a capillary member can have a narrow part for insertion into a reservoir, leading to a wider evaporation surface, the member typically having a shape resembling a small tennis racquet. This capillary member does not suffer from the fractionation effects of porous wicks.

The problem with currently-known capillary members is that the available patterns and configurations have proved ineffective at ensuring efficient evaporation. For example, in the case of a sheet, individual channels spreading radially from the transfer member become further and further apart, and only a small portion of the possible evaporative surface is utilised. A suggestion for overcoming this has been a cross-hatched surface, that is, a series of parallel channels is crossed by another series of parallel channels. While in theory this allows liquid to flow to the entire surface, practically this is not the case, as it has been found that the volatile liquids show a marked reluctance not only to flow into a channel (“secondary channel”) crossing the channel they are in, but also to cross the channel intersection and continue along the original (“primary”) channel. In the case of a capillary member comprising an evaporating surface on which is a series of capillary channels that is inserted directly into a liquid reservoir, only those channels in contact with the liquid will make up the evaporative surface, which will thus be limited by the amount of capillary rise up these elements.

It has now been surprisingly found that this problem can be at least substantially overcome and evaporation from a substantial portion of the surface area of a capillary member can be assured by a simple and inexpensive construction. The invention therefore provides an apparatus adapted to disseminate a volatile liquid into an atmosphere from a reservoir, dissemination being achieved by means of a transfer member in contact with the liquid and a capillary member in liquid transfer contact with the transfer member, the capillary member comprising an evaporating surface bearing primary capillary channels, at least some of these primary capillary channels being intersected by at least one secondary capillary channel, the secondary capillary channel being substantially smaller in cross-sectional area than that of the primary channels, such that liquid will flow in both primary and secondary channels.

The invention additionally provides a method of disseminating a volatile liquid to an atmosphere by evaporation from an evaporating surface, comprising conveying the liquid from a reservoir by means of a transfer means to the evaporating surface in liquid transfer contact therewith, the evaporating surface comprising primary capillary channels, at least some of these primary capillary channels being intersected by at least one secondary capillary channel, the secondary capillary channel being substantially smaller in cross-sectional area than that of the primary channels, such that liquid will flow in both primary and secondary channels.

With such a structure, it has been surprisingly found that the liquid not only flows across the intersection readily to continue along the channel, but it also flows into the intersecting channel. Thus, the provision of appropriately-placed and -sized secondary channels ensures that substantially the entire evaporating surface may be used.

The evaporating surface is a surface whose length and breadth are appreciably larger than its thickness, which surface bears capillary channels. The shape of the surface is not significant, and it can be chosen from any suitable decorative or practical shape. The capillary channels may be on one or both sides of the surface. The surface and its capillary channels may be provided by any convenient means, for example, by injection moulding or engraving.

The transfer member may be any such member suitable for transferring volatile liquid from a reservoir to an evaporating surface. For example, it may be a porous wick of the type well known to the art, and it may be made of any suitable material, such as cellulose, graphite or ceramic material. In such a case, the capillary evaporating surface extends substantially perpendicularly from the wick, and it may be attached thereto by any convenient means. For example, the wick may be supplied with an annular groove into which the capillary member fits. It may also comprise a slot, which mates with a matching tab in the capillary member. A further possibility is a transfer member that is frusto-conical, that is, it tapers slightly as it extends away from the reservoir. This allows for easy mounting of a capillary sheet that has a mounting hole intermediate in diameter between the maximum and minimum diameters of the transfer means. The skilled person will, by the exercise of the ordinary skill of the art, readily be able to devise further variants lying within the scope of the invention.

Alternatively, the transfer member may comprise capillary channels in the manner of U.S. Pat. No. 4,913,350. In this case, the evaporating surface may simply comprise a broadening of the transfer member at its upper end, often resulting, as previously mentioned, in a shape similar to that of a small tennis racquet.

An alternative capillary transfer member may be provided by a gap of capillary proportions formed at the junction of two flat surfaces. This leads to the capillary channels of the evaporating surface.

By “capillary channels” is meant channels open to the atmosphere and of such dimensions that capillary flow will occur within them. Provided this happens, the channels can be any suitable shape and dimension, and suitable dimensions for any given application may readily be determined by simple experiment. Typical primary capillary channels are of “V” shaped cross section and have dimensions of 0.1-0.5 mm width at the top, 0.1-0.5 mm depth with the “V” angle of the channel being 10-25 degrees. Preferred primary channels are approximately 0.2 mm wide at the open top, approximately 0.4 mm vertically deep and have an angle of approximately 24 degrees.

At least some of the primary channels are intersected by at least one secondary channel. Preferably all primary channels are intersected by the at least one secondary channel, more preferably by a multiplicity of secondary channels. The object is to provide capillary channels covering a large surface area, and the precise pattern or arrangement is not critical.

Although it is preferred that liquid transfer contact be between the transfer member and the primary channels (from which most evaporation takes place), this is not necessary, and it is possible to use at least one secondary channel to transfer the liquid from the transfer member to the primary channels.

The secondary channel or channels is or are of substantially smaller cross-sectional area than that of the main channels. Because of the varying natures of volatile liquids, what exactly constitutes “substantially smaller” will differ, sometimes considerably, from case to case. However, given the concept that flow across the entire surface is a result of the use of two different channel sizes, the skilled person can readily determine for each liquid and each dimension of main channel what constitutes “substantially smaller” in respect of the intersecting channel. The secondary channels are generally (and preferably) substantially narrower than the primary channels. As a guide (and without limiting the invention in any way), typical dimensions for the release of a fragrance are, for a secondary channel, a depth of 0.05 mm. and a cross-sectional area of less than 90%, preferably less than 50%, of that of a primary channel. It is not necessary that the secondary channel be as deep as the primary channel, but it should naturally be sufficiently deep that liquid can flow into it. Preferably, both primary and secondary channels are of equal depth. However, it is possible and permissible that the secondary channels be deeper than the primary channels.

As hereinabove mentioned, the particular pattern of channels is not critically important and a wide variety is possible. For example, a pattern of primary channels may extend radially from the contact with the transfer member and the secondary channels may form a series of concentric circles, extending out to the edge of the sheet. Another possibility is a series of parallel primary channels intersected by at least one other series of parallel secondary channels.

In a very simple embodiment, a series of primary channels may be parallel grooves and the secondary channel may be at least one thin slit cut into or completely through the evaporating surface. In a further embodiment of this type, the evaporating surface may comprise a number of sections that fit together, the boundaries where they meet providing the desired slit or slits. In such a case, the primary channel pattern may be optimised. For example, the sheet may be in four equal portions that fit tightly into a holding plate, and the primary channels in each portion may extend in parallel fashion away from the transfer member, the assembled sheet having a “herringbone” appearance.

In a further embodiment of the invention, the primary capillary channels may extend to the end of the sheet, and there be blocked by a transverse barrier, such as a wall, placed across the open channel ends, so as to define a secondary capillary channel that allows liquid to flow between channels.

The capillary sheets of this invention may be made of any suitable material that is not affected by the volatile liquid over the desired lifetime of the sheet. The channels therein may be provided by any convenient means, for example, by injection molding or by engraving.

The invention is further described with reference to the drawings, which depict preferred embodiments and which are not limiting on the scope of the invention.

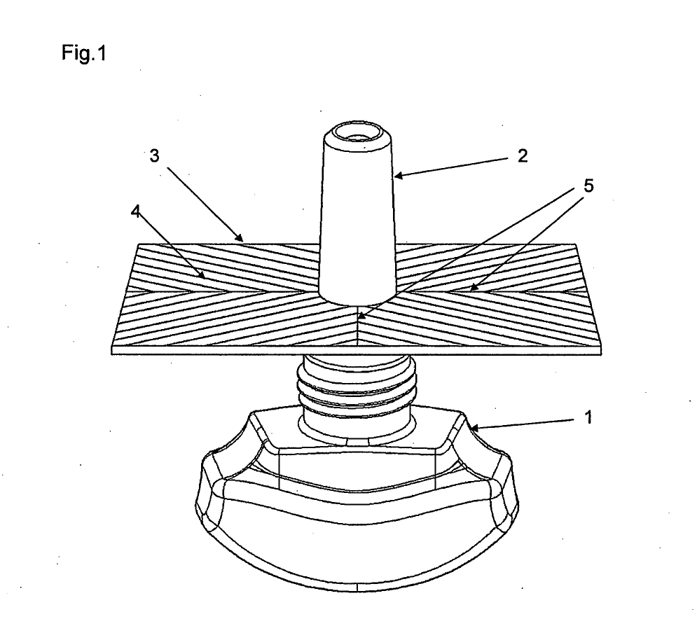

FIG. 1 is a perspective view of an embodiment of the invention.

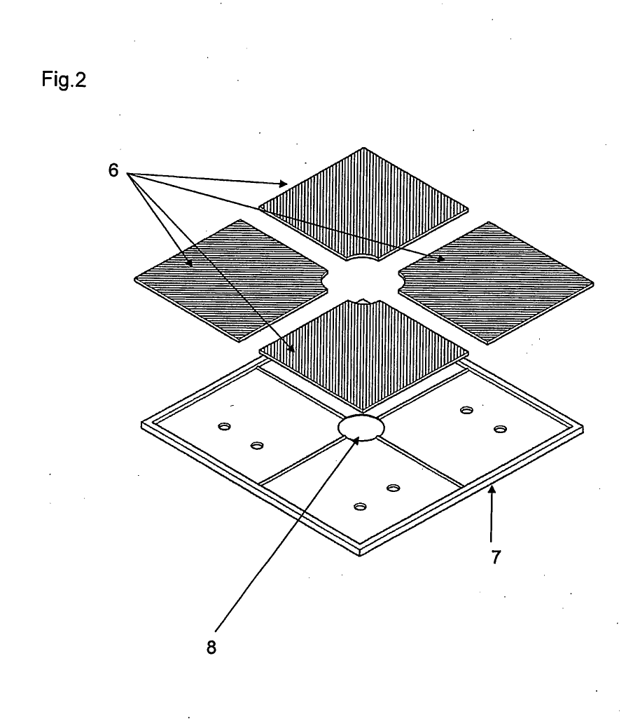

FIG. 2 is a perspective, exploded view of an evaporating surface for use in the embodiment of FIG. 1.

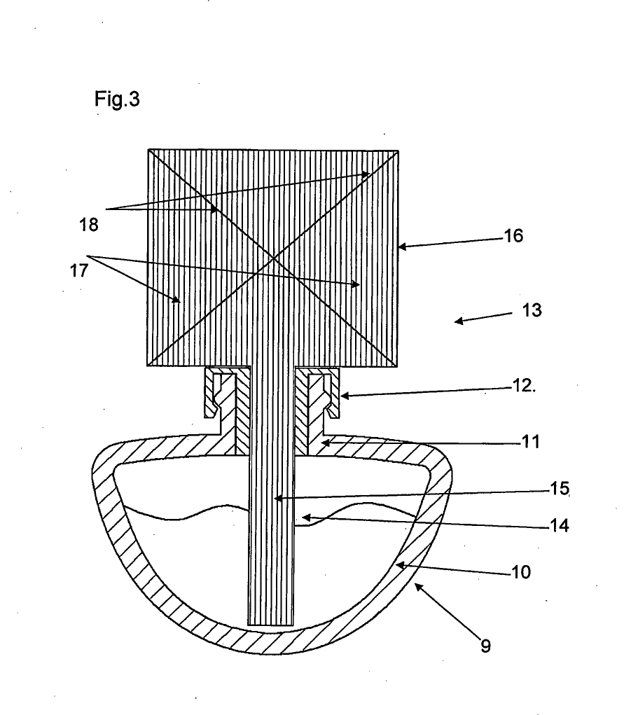

FIG. 3 is a longitudinal cross-section of a further embodiment of the invention.

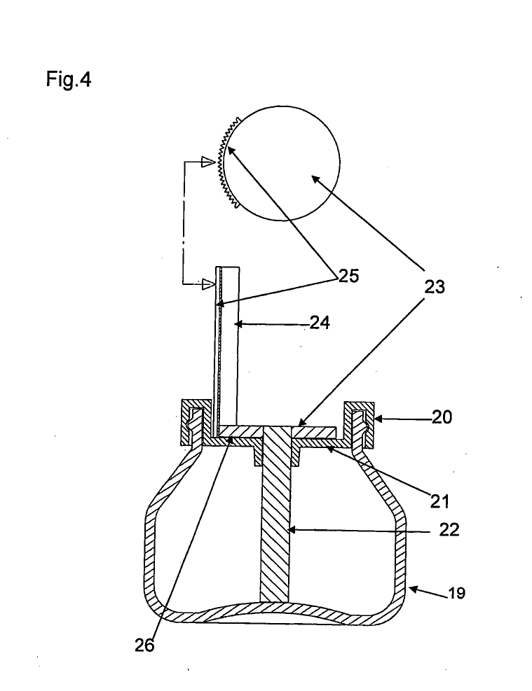

FIG. 4 is a longitudinal cross-section of a further embodiment of the invention, with additional detail of one aspect.

In FIG. 1, a reservoir 1 contains volatile liquid, to be disseminated into an atmosphere. This liquid leaves the reservoir by means of a porous wick 2. Fitted around this porous wick is an evaporation surface 3, bearing on this surface series of capillary channels 4. The evaporation surface 3 is actually made up of four plates, which fit tightly together, meeting at joins 5, which are relatively narrow in comparison with the capillary channels 4.

A better comprehension of the evaporating surface can be gained by studying FIG. 2. Here can be seen the four quarter-plates 6 that make up the evaporation surface. These fit into a holding plate 7, which has a centrally-located hole 8, to permit mounting on the wick 2. Those parts of the quarter-plates 6 that contact the wick 2 are shaped so that they fit sufficiently tightly around the wick such that the capillary channels on the quarter-plates are in liquid transfer contact with the wick. In this embodiment, the capillary channels of the individual quarter-plates are parallel to each other, the central radius of each quarter-plate being radial to the wick. The four thus form a “herringbone” pattern. The joints 5 between the quarter-plates 6 form secondary capillary channels, and they convey liquid to primary channels that are not in direct contact with the wick. In addition, the gaps formed where the edges of the holding plate and the quarter-plates also act as secondary channels and convey liquid. The liquid can therefore be disseminated over the entire evaporating surface.

The embodiment shown in FIG. 3 comprises a reservoir 9 containing a volatile liquid 10. Into a neck 11 of the reservoir is fitted a stopper 12. Through this stopper goes an evaporating surface and transfer member, generally indicated as 13. The transfer member 14, through which liquid is drawn from the reservoir is a flat capillary member 14 having capillary channels 15. The transfer member extends into a flat planar evaporating surface 16. This evaporating surface not only includes the continuation of the capillary channels 15 of the transfer member 14, but also additional capillary channels 17 parallel to those channels. In this case, the evaporating surface is square (although it can be any desired functional or decorative shape), and it additionally bears diagonally-extending secondary channels 18 of smaller size than the capillary channels 15 and 17, which allows the liquid to flow into all the capillary channels, including those not in direct connection with the liquid.

In the embodiment of FIG. 4, liquid is transferred from a liquid transfer member by means of a secondary channel. A reservoir 19 is fitted with a cap 20 that has a planar closure 21 extending across the open neck. Through a hole in this closure fits a cylindrical wick 22, which leads to a planar diffusion member 23. The wick sits on the bottom of the reservoir, such that the planar diffusion member does not rest on the planar closure, but is supported slightly above it, to define a secondary capillary channel 26 between the two. The diameter of the planar diffusion member 23 is less than that of the internal diameter of the cap 20, leaving an annular space around the edge of the diffusion member. Into this is fitted a curved plate 24 that bears primary capillary channels 25 on its outwardly-facing face.

In operation, a liquid moves from the reservoir via the wick 22 to the secondary capillary channel 26. It moves along this channel until it reaches the ends of the primary capillary channels 25. It then moves up these channels and evaporates into the atmosphere.

1. An apparatus adapted to disseminate a volatile liquid into an atmosphere from a reservoir, dissemination being achieved by means of a transfer member in contact with the liquid and a capillary member in liquid transfer contact with the transfer member, the capillary member comprising an evaporating surface bearing primary capillary channels, at least some of these primary capillary channels being intersected by at least one secondary capillary channel, the secondary capillary channel being substantially smaller in cross-sectional area than that of the primary channels, such that liquid will flow in both primary and secondary channels.

2. An apparatus according to claim 1, in which the transfer member is a substantially cylindrical porous wick and the evaporating surface is a sheet extending substantially perpendicularly from the wick, the sheet bearing capillary channels on at least one surface.

3. An apparatus according to claim 1, in which the transfer member is a capillary member that extends from the liquid to the evaporating surface.

4. An apparatus according to claim 3, in which the transfer member and the evaporating surface are formed in one piece.

5. An apparatus according to claim 1, in which the transfer member is a gap of capillary proportions formed at the junction of two flat surfaces.

6. An apparatus according to claim 1, in which the evaporating surface comprises a number of sections bearing primary capillary channels, the boundaries where they meet providing the secondary channel or channels.

7. An apparatus according to claim 6, in which the primary capillary channels in each section are parallel and extend away from the transfer member, the assembled sheet having a “herringbone” pattern.

8. An apparatus according to claim 1, in which the primary capillary channels extend to the end of the sheet and are there blocked by a transverse barrier placed across the open channel ends, so as to define a secondary capillary channel that allows liquid to flow between channels.

9. An apparatus according to claim 1, in which at least one secondary channel transfers the liquid from the transfer member to the primary channels.

10. A method of disseminating a volatile liquid to an atmosphere by evaporation from an evaporating surface, comprising conveying the liquid from a reservoir by means of a transfer means to the evaporating surface in liquid transfer contact therewith, the evaporating surface comprising primary capillary channels, at least some of these primary capillary channels being intersected by at least one secondary capillary channel, the secondary capillary channel being substantially smaller in cross-sectional area than that of the primary channels, such that liquid will flow in both primary and secondary channels.
